(19) United States
(12) Patent Application Publication
(10) Pub. No.: US 2026/0124352 A1
(43) Pub. Date: May 7, 2026

(54) METHODS AND SYSTEMS FOR TREATMENT OF CEREBRAL EDEMA

(71) Applicant: Inventx LLC, Roseville, MN (US)

(72) Inventors: Rick M. Odland, Roseville, MN (US); Michael R. Wilson, Lexington, KY (US); John Borgos, Shoreview, MN (US)

(73) Assignee: Inventx LLC, Roseville, MN (US)

(21) Appl. No.: 18/937,732

(22) Filed: Nov. 5, 2024

Related U.S. Application Data
(60) Provisional application No. 63/591,935, filed on Oct. 20, 2023.

(51) Int. Cl. A61M 1/00; A61B 17/34

(52) U.S. Cl. CPC A61M 1/71; A61B 17/3415; A61M 2202/0014; A61M 2202/0464; A61M 2205/04; A61M 2210/0693

(57) ABSTRACT

Methods and systems for reduction of cerebral edema including catheter systems. A catheter system includes an elongated main body including a proximal end, a distal end, a central lumen and a plurality of outer lumens extending therethrough and the outer lumens positioned around the central lumen, a hollow fiber portion distal to the main body, and a cap distal to and connected to the hollow fibers. The hollow fiber portion includes a plurality of hollow fibers comprising elongated hollow tubes with semipermeable walls surrounding a drainage catheter. The hollow fibers terminate in the cap which forms a plenum for gas sweeping through the outer lumens and the hollow fibers.

### CROSS REFERENCE TO RELATED APPLICATION

This application claims priority to U.S. provisional application 63/591,035, filed Oct. 20, 2023, entitled Improved Methods and Systems for Treatment of Cerebral Edema, the disclosure of which is hereby incorporated by reference.

### BACKGROUND

Cerebral edema may be caused by traumatic brain injury (TBI) due to impacts such as motor vehicle accidents, falls, and sports injuries. Other causes of cerebral edema include, for example, brain tumors and infections. In many other areas of the body, tissue swelling may be relatively harmless. However, because the brain is contained within the boney structure of the skull, cerebral edema causes an increase in intracranial pressure (ICP). Because of the fragile nature of brain tissue, elevated ICP can damage the cells of the brain, resulting in permanent brain injury or even death. Therefore, effective treatments to reduce ICP are essential and potentially lifesaving.

One method of reducing ICP is removal of a portion of the skull through a craniotomy procedure, thereby providing room for the brain to expand. However, while a craniotomy does reduce ICP, the clinical outcomes for patients are less than desired.

Other treatments include hyperosmolar agents. Systemic agents such as mannitol and hypertonic saline are provided to patients intravenously, such as through bolus injections. These hyperosmolar agents cause water movement out of the brain and reduce cerebral edema to a certain extent, but their impact is limited. Other efforts have focused on direct infusion of hyperosmolar agents into the cerebral ventricles, but infusion into the ventricles tends to raise ICP, especially in the noncompliant injured brain.

Another method to reduce cerebral edema is to drain off cerebrospinal fluid (CSF) using an External Ventricular Drainage (EVD) catheter. In this method, a catheter is inserted into the ventricle to drain the CSF. In this way, the EVD catheter can successfully reduce the ICP, but the effect may be temporary and, as with other methods, it is unclear how much it improves patient outcomes. Reduction of hydrostatic backpressure by draining ventricular CSF may even worsen cerebral edema.

Improved devices and methods are, therefore, needed to reduce cerebral edema as well as intracranial pressure in order to make progress in improving patient outcomes.

### SUMMARY

Various embodiments include catheters system for reduction of cerebral edema comprising a catheter. In some embodiments, the catheters include an elongated main body comprising a proximal end, a distal end, a central lumen and a plurality of outer lumens, the central lumen and the outer lumens extending therethrough and the outer lumens positioned around the central lumen. They also include a hollow fiber portion distal to the main body including a plurality of hollow fibers comprising elongated hollow tubes with semipermeable walls, the semipermeable walls being gas permeable and liquid impermeable, the hollow fibers each comprising a proximal end connected to one of the plurality of outer lumens, and a drainage catheter including a central lumen, a plurality of pores, and having a proximal end connected to the central lumen of the elongated main body and a distal sealed end, with the plurality of hollow fibers are positioned around the drainage catheter. They also include a cap distal to and connected to the hollow fibers, the cap comprising an outer wall that forms a central space into which the hollow fibers extend and terminate at open distal ends, the central space forming a plenum for gas sweeping through the outer lumens and the hollow fibers.

In some embodiments, the catheter system of claim 1, the catheter also includes a manifold proximal to the main body, and the outer lumens split into a first group and a second group lateral to the central lumen. The manifold may have an oval cross sectional shape. The catheter system may also include a drainage catheter extension extending proximally from the manifold. In some embodiments, the catheter system may also include a trocar having an elongated main body, a sheath, and a connector end, with the connector end configured to connect to the drainage catheter extension of the catheter. In some such embodiments, the sheath is slidably engaged with the trocar main body and configured to slide over and around the manifold of the catheter when the catheter and the trocar are connected.

In some embodiments, the hollow fibers have an outer diameter between about 100 and about 500 micrometers. The semipermeable walls of the hollow fibers may include micropores and a permeable material extending over the micropores.

In some embodiments, the catheter system includes a trocar having an elongated main body with a first end and a second end, the first end comprising a tip and the second end comprising a connector configured to connect to the proximal end of the catheter main body, and a sheath surrounding the main body and slidable on the main body. The sheath includes a neck portion surrounding a portion of the main body and having a circular cross-sectional shape, a shoulder portion which flares outward in one plane to form an expanding oval cross sectional shape, and a tubular sleeve portion extending around and spaced apart from the main body and having a uniform oval cross sectional shape, with the shoulder portion located between the neck portion and the tubular sleeve portion. In some embodiments, the shoulder portion flares outward at an angle of between about 30 degrees and about 60 degrees. In some embodiments, the connector is configured to fit snugly within the central lumen of the catheter main body. For example, in some such embodiments, the connector comprises a series of tapered barbs. In some embodiments, the elongated main body of the trocar comprises two straight portions connected at an angle. The sheath may be located between the angle and the connector of the main body, for example.

Various embodiments include methods of treatment of cerebral edema. The methods include, for example, inserting a distal end of a catheter into a ventricle of a brain of a patient suffering from cerebral edema. The catheter may include an elongated main body comprising a proximal end, a distal end, a central lumen and a plurality of outer lumens, the central lumen and the outer lumens extending therethrough and the outer lumens positioned around the central lumen, a hollow fiber portion distal to the main body, the hollow fiber portion comprising a plurality of hollow fibers comprising elongated hollow tubes with semipermeable walls, the semipermeable walls being gas permeable and liquid impermeable, the hollow fibers each comprising a proximal end connected to one of the plurality of outer lumens, and a drainage catheter, and the drainage catheter comprising a central lumen, a plurality of pores, the drainage catheter having a proximal end connected to the central lumen of the elongated main body and a distal sealed end, the plurality of hollow fibers are positioned around the drainage catheter, a cap distal to and connected to the hollow fibers, the cap comprising an outer wall that forms a central space into which the hollow fibers extend and terminate at open distal ends, the central space forming a plenum for gas sweeping through the outer lumens and the hollow fibers, and a manifold connected to the proximal end of the main body, wherein the outer lumens split into a first group and a second group lateral to the central lumen. The method may further include connecting the catheter to a source of dry gas and a vacuum, the dry gas supplied to the catheter through the first group of outer lumens and the vacuum applied to the catheter through the second group of outer lumens and supplying the dry gas and vacuum to the catheter. In some embodiments, the dry gas and the vacuum are supplied continuously. In other embodiments, the dry gas and the vacuum are supplied intermittently, such as in an optimized removal schedule. Some embodiments also include draining cerebrospinal fluid from the ventricle through the central lumen of the main body.

Various embodiments include methods of inserting a treatment catheter into a treatment location in a patient. For example, the method may include forming a craniotomy hole at a location in the skull of the patient, inserting a first end of a treatment catheter through the craniotomy hole and to a position for treatment, the treatment catheter further including a second end which remains external to the patient after this step of inserting the first end, forming a tunnel beneath the scalp of a patient, the tunnel extending from the location of a craniotomy hole to an incision in the scalp at a distance of 2 centimeters or more from the craniotomy hole location, after inserting the first end of the treatment catheter through the craniotomy hole into the position for treatment, attaching the second end of a treatment catheter to a connector end of a trocar, passing the trocar with the attached treatment catheter through the tunnel such that the trocar pulls the second end of the treatment catheter through the tunnel and the second end of the treatment catheter exists the tunnel through the incision, and then detaching the trocar from the treatment catheter. The second end of the catheter may include an external aperture connected to a catheter lumen and sealed with a removable plug, and after the second end of the treatment catheter exists the tunnel through the incision, the method includes removing the plug from the aperture.

### BRIEF DESCRIPTION OF THE DRAWINGS

The following drawings are illustrative of particular embodiments and do not limit the scope of the inventions. The drawings are not necessarily to scale and are intended for use in conjunction with the following detailed description. Embodiments of the inventions will be described with reference to the drawings, in which like numerals may represent like elements.

FIG. 1 is a depiction of water movement during a reductive ventricular osmotherapy treatment;

FIG. 2 is a representation of a catheter system according to various embodiments;

FIG. 3 is a representation of a catheter system in place in a brain according to various embodiments;

FIG. 4 is a photograph of a catheter according to various embodiments;

FIG. 5 is diagram of a distal end of a catheter according to various embodiments;

FIG. 6 is a diagram of a proximal end of a catheter according to various embodiments;

FIG. 7 is a diagram of a catheter in a top view and side view and of a style for use with the catheter according to various embodiments;

FIG. 8 is a diagram of example components and controls in a control module according to various embodiments;

FIG. 9 is a photograph of a front of a control module according to various embodiments;

FIG. 10 is a photograph of the back of the control module of FIG. 9;

FIG. 11 is a photograph of a catheter containing a stylet according to various embodiments;

FIG. 12 is a photograph of the catheter and stylet of FIG. 11 with the stylet removed from the catheter;

FIG. 13 is a diagram of a trocar according to various embodiments;

FIG. 14 is a photograph of a sleeve portion of a trocar according to various embodiments;

FIG. 15 is a photograph of a trocar in proximity to a proximal end of a catheter according to various embodiments;

FIG. 16 is a photograph of the trocar and proximal end of the catheter of FIG. 4 being connected together;

FIG. 17 is a photograph of the trocar and proximal end of the catheter of FIG. 4 connected together;

FIG. 18 is a diagram of a proximal end of a catheter according to various embodiments;

FIG. 19 is diagram of a proximal end of the catheter of FIG. 18 with the plugs replaced by Luer connectors;

FIG. 20 is diagram of the proximal end of the catheter of FIGS. 18 and 19 with airflow lines connected to the Luer connectors;

FIG. 21 is a photograph of a catheter connected to air supply lines according to various embodiments

FIG. 22 is a graph of change in osmolality over time for RVOT at 50% and 100% intensity;

FIG. 23 is a graph of change in osmolality over time when RVOT is continued verses discontinued;

FIG. 24 is a graph of change in osmolality over time for RVOT at 50% intensity at the beginning of treatment;

FIG. 25 is a graph of change osmolality over time for RVOT at 50% for four hours;

FIG. 26 is a graph of change in osmolarity over time at 50% RVOT intensity;

FIG. 27 is a graph comparing change in osmolarity over time for two different on/off cycle times at 25% RVOT intensity; and

FIG. 28 is a graph comparing change in osmolarity over time for two different on/off cycle times at 12.5% RVOT intensity.

### DETAILED DESCRIPTION

The following detailed description is exemplary in nature and is not intended to limit the scope, applicability, or configuration of the inventions. Rather, the following description provides practical illustrations for implementing various exemplary embodiments. Utilizing the teachings provided herein, those skilled in the art may recognize that many of the examples have suitable alternatives that may be utilized.

Various embodiments include improved methods and systems for using catheter systems including porous, semiporous, or water impermeable hollow fibers to remove water vapor from the ventricles in a process referred to herein as Reductive Ventricular OsmoTherapy (RVOT). The catheter systems include an array of hollow fibers at a distal end of the catheter which is inserted into the ventricle of the brain. A dry sweep gas is passed through the hollow fibers, removing water vapor from the CSF within the ventricles. This, in turn, increases the osmolality of the CSF which draws water out of the brain cells, thereby reducing cerebral edema. As the RVOT system continues to remove water vapor from the CSF, the CSF continues to draw water out of the cells. Removal of water vapor also reduces the volume of intracranial water. The RVOT system is uniquely able to simultaneously increase CSF osmolality while reducing intracranial volume. In this way, the methods and systems described herein reduced ICP as well as cerebral edema. The catheter system may further include a drainage lumen for bulk removal of CSF from the ventricle. In this way, the system can provide both water vapor removal using the hollow fibers and/or bulk CSF drainage using the drainage lumen.

The hollow fibers used in various embodiments described herein may be thin walled elongated hollow tubes with semipermeable walls which make them gas permeable and liquid impermeable. The hollow fibers may have any length, but in various embodiments described herein they are sized for removal of fluid from the lateral ventricles of the brain and may be between about 1 and about 6 cm in length, such as about 2 to about 4 cm in length, or about 3 cm to about 4 cm in length. The hollow fibers have a very small, uniform outer diameter, such as between about 100 to about 500 micrometers, or about 200 to about 400 micrometers, or about 300 micrometers.

The hollow fibers may be made of a thin main body material having micropores. Examples of materials which may be used for the main body include hydrophobic membranes such as hydrophobic polypropylene membranes. The micropores may be 0.2-0.4 microns, for example. When the pore is coated, the hollow fiber material may be hydrophobic or hydrophilic. The outer surface of the main body may be coated with an ultrathin layer of a permeable material which extends over the micropores and over the entire length of the hollow fiber. An example of such a coating material is silicone such a cross linked silicone having the same chemical moieties and chemical properties as medical grade silicone. The ultrathin layer may be less than 10 micrometers thick, such as about 0.5 to about 2 micrometers, such as about 1 micrometer thick, for example. In this way, the material of the main body material is sufficiently strong to form an elongated internal passage and prevent the hollow fiber tube from collapsing, while providing pores that would allow fluids to pass into the central hollow passage of the hollow fibers. However, the coating that extends over the pores restricts the passage of material into the pores. The composition of the coating and the extremely thin nature of the coating prevents the movement of water or solutes into the hollow fibers while still allowing the movement of water vapor into the hollow fibers.

In various embodiments, the distal end of the RVOT catheter includes a plurality of hollow fibers which are placed into the CSF in the lateral ventricle of the brain. Through the catheter, a first plurality of the hollow fibers are connected to a gas which is supplied to the catheter at the proximal end, while a second plurality of hollow fibers are connected to a vacuum source at the proximal end of the catheter. A plenum at the distal tip of the catheter allows the incoming gas to sweep through the first plurality of hollow fibers, through the plenum, and then out through the second plurality of hollow fibers through the negative pressure of the vacuum. When a dry gas is used, the dry gas sweeps through all of the hollow fibers and becomes hydrated by water vapor from the CSF. This gas may be referred to as sweep gas.

During an RVOT procedure, water vapor pressure drives water vapor through the silicone coating of the hollow fibers and into the lumen of the hollow fiber in a process of pervaporation. As the dry gas sweeps through the hollow fibers, the water vapor is carried away to allow steady removal of water from the CSF. RVOT water vapor removal from the CSF causes an increase in CSF osmolarity. The creation of hyperosmolar CSF results in an osmotic gradient between the CSF and the cells of the brain. This osmotic gradient induces the movement of water out of the tissue and into CSF in the ventricular space. This water can then be removed by the continued flow of dry gas through the hollow fibers in the CSF as the RVOT procedure continues. In this way, the gradual removal of water vapor from the CSF gradually pulls water from the cells of the brain. Reduction of cellular swelling opens up intracellular pathways for improved convective interstitial flow. Removal of water vapor simultaneously reduces intracranial volume, reducing both cerebral edema by an osmotic mechanism and intracranial pressure by osmotic and convective mechanisms.

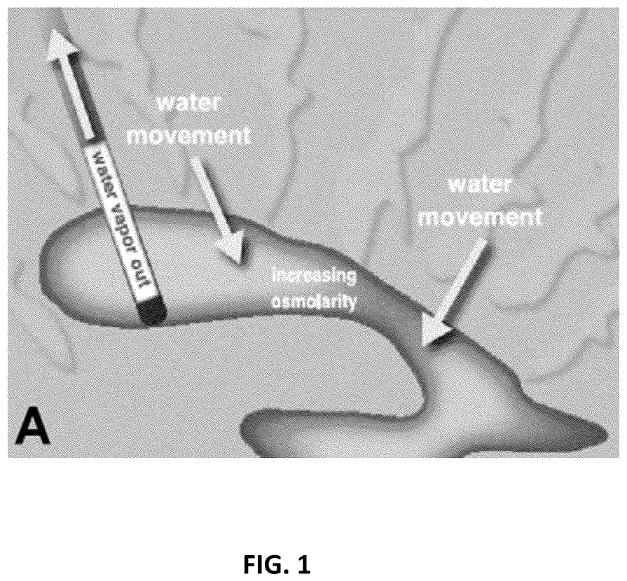

FIG. 1 shows a representative example of how water vapor may be removed from the ventricle using the hollow fiber catheter system according to various embodiments. The distal end of the catheter is located in the lateral ventricle where it removes water vapor from the CSF. This increases the osmolarity of the CSF, causing water movement out of the brain cells.

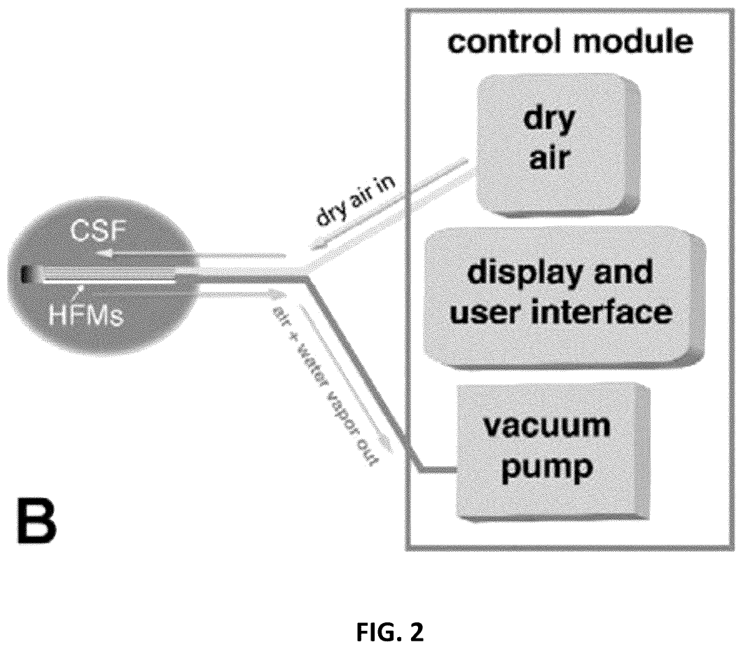

FIG. 2 shows a representative example of how dry gas is swept through the hollow fibers at the distal end of the RVOT catheter under the control of a control module. The control module controls the supply of dry gas to the proximal end of the catheter, and also includes and controls a vacuum pump that pulls the dry gas through the catheter.

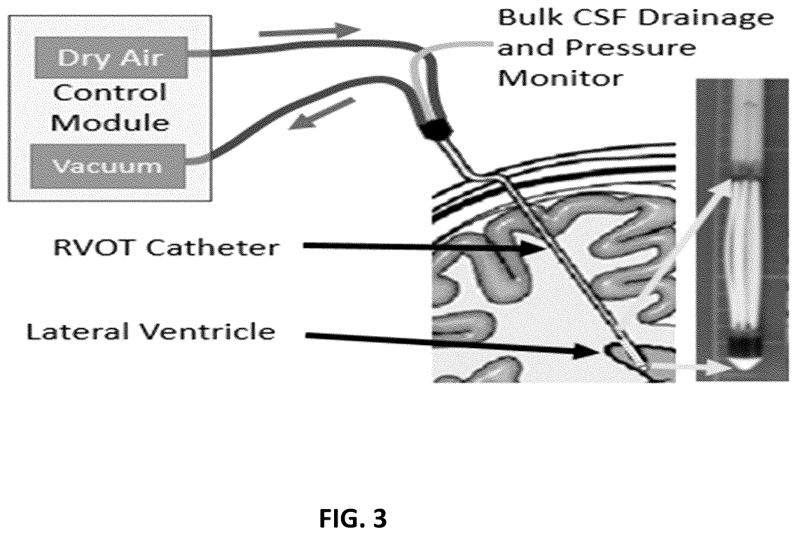

A further representative example is shown in FIG. 3, in which the catheter has been inserted through the cranium and into the lateral ventricle, with the distal end of the catheter including the exposed hollow fibers in the lateral ventricle. At the proximal end, the control module supplies and controls dry air which is swept through the catheter and the hollow fibers. The catheter further includes a lumen for connection to a vacuum for removal of bulk CSF from the ventricle, which can occur separately from or simultaneously with the water vapor removal through the hollow fibers.

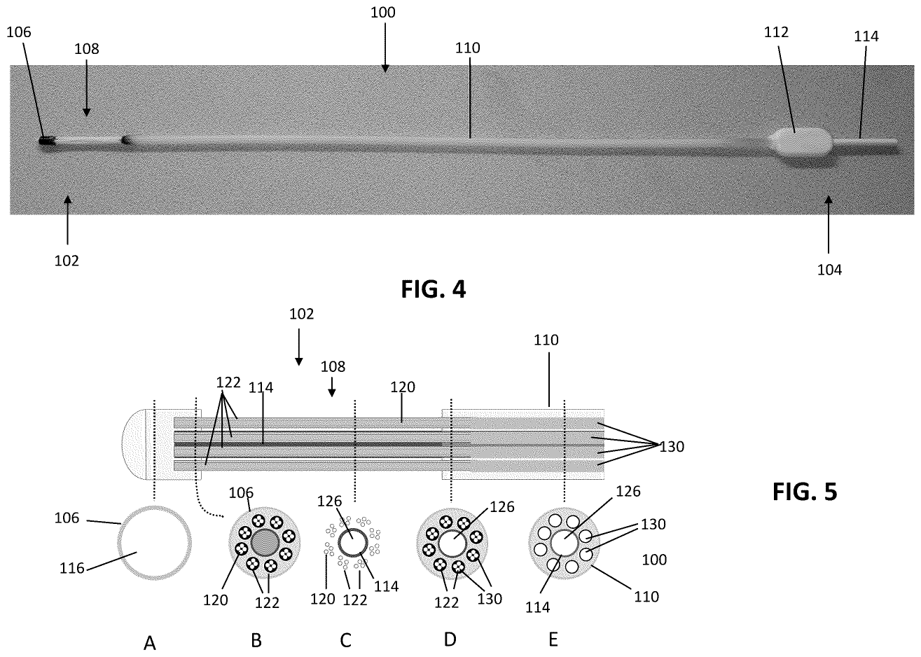

FIG. 4 shows an example of a catheter 100 which may be used in various embodiments. The catheter includes a distal end 102 and a proximal end 104. A cap 106 at the distal end 102 provides a plenum for gas circulation. Immediately proximal to the cap 106 is the hollow fiber section 108. Immediately proximal to the hollow fiber section 108 is the main body 110. Immediately proximal to the main body 110 is the manifold 112. At the proximal end of the catheter, the CSF bulk drainage line 114 extends proximally from the manifold. In use, after placement of the hollow fiber section 108 of the catheter 100 in the desired location, the proximal end 104 of the catheter 100 is connected to external devices such as a controller (also referred to as a control module) through air line extension sets for inflow and outflow of the sweep gas. During use, the central lumen 114 may be connected to a standard, commercially available CSF drainage system.

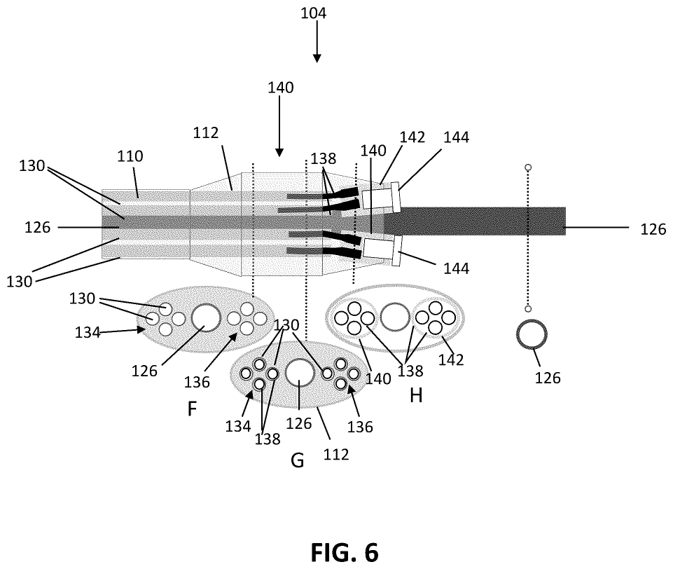

FIGS. 5 and 6 show a more detailed view of the catheter distal and 102 (FIG. 5) and proximal end 104 (FIG. 6), including cross sections at various locations to better reveal the system for airflow and fluid removal.

As shown in FIG. 5, the cap 106 is located at the distal tip of the catheter 100. Cross section A shows that the cap 106 includes an outer wall that surrounds and forms a hollow central space, the plenum 116, into which each of the hollow fibers 120 connect at their distal ends. As described further below, half of the hollow fibers 120 are connected to a supply of gas, while the other half are connected to a vacuum. The plenum 116, therefore, allows for gas to pass between the hollow fibers 120. That is, gas flows out of the distal ends of the hollow fibers 120 into which gas is supplied, into the plenum 116, and then exits the plenum 116 through the hollow fibers 120 through which a vacuum is applied.

Cross section B shows the connection between the hollow fiber section 108 and the cap 106. The distal ends of the hollow fibers 120 extend into the proximal end of the cap 106, where they end. The distal ends of the hollow fibers are affixed within the cap 106 using an airtight and secure sealing method such that no air can escape from the plenum 116 and into the ventricle of the brain. At the same time, it is important that the sealing method not obstruct airflow through the distal ends of the hollow fibers.

In the hollow fiber section 108, the outer surfaces of the hollow fibers 120 are exposed to the surrounding environment providing a large surface area for water vapor pervaporation. When in place in the lateral ventricle, the hollow fibers will be bathed in CSF. In the example shown in FIG. 5, the hollow fibers are arrayed in a ring formation. However, the hollow fibers 120 are not uniformly distributed around the circumference, such as in a single layer or multiple players. Rather, as can be seen in cross section C, the hollow fibers 120 are in clusters 122 of 4 hollow fibers 120 each, and these clusters 122 are arrayed in a ring formation around the circumference of the catheter hollow fiber section 108 of the catheter 100. It can further be seen that, while the hollow fibers 120 of each cluster 122 are in close proximity to each other and potentially abutting each other, there is a small gap between adjacent clusters 122, such that the space between the hollow fibers 120 of adjacent clusters 122 is larger than the spaces between the hollow fibers 120 of a particular cluster 122.

The hollow fiber section may be between about 2 centimeters and about 4 centimeters in length, such as about 3 centimeters in length, for example. Other lengths are also possible.

In the example shown, the clusters 122 include four hollow fibers 120 each and there are 8 clusters 122, for a total of 32 hollow fibers 120. This arrangement of hollow fibers 120 fits within the approximately 3-4 mm outer diameter of the catheter 100. In alternative embodiments, the clusters 122 could include few or more hollow fibers 120, such as 2-8 fibers 120, or 3-6 or 3-5 fibers 120. Likewise, the catheter 100 could include fewer or more clusters 122, such as 6-10 clusters 122, or 7-9 clusters 122. However, it is important to maximize the exposed surface area of the hollow fibers 120 to maximize the water vapor transmission. That is, the hollow fibers 120 of each cluster 122 should have a large outer surface area which is exposed to the environment, and the surface area which is abutting an adjacent hollow fiber 120 should minimized. The use of a large number of hollow fibers 120 increases the total exposed outer surface area. At the same time, it is important that the diameter of the catheter 100 be kept small, and as such the total number of hollow fibers 120, and hollow fiber clusters 122, and the number of hollow fibers 120 in each cluster 122, needs to be considered and controlled. The embodiment showin including eight clusters 122 of hollow fibers 120, each including four hollow fibers 120, is one useful example for maximizing the exposed surface area of each hollow fiber 120 while maintaining a small circumference of the catheter 100.

The hollow fiber section 108 further includes a centrally located drainage catheter 144, surrounded by the hollow fiber clusters 122. The drainage catheter 114 includes a plurality of pores to allow bulk drainage of CSF through the pores and into the central lumen 126 of the drainage catheter. As can be seen in cross section C, the drainage catheter 114 is hollow with a central lumen 126 extending through its length. The drainage catheter 114 can be connected to a bulk drainage system at its proximal end as needed for bulk fluid removal. In cross section B, the distal end of the drainage catheter 114 is affixed to the cap 106. However, unlike the hollow fibers 120, the distal end of the drainage catheter 114 is not open into the cap 106 but rather the central lumen is sealed to prevent fluid entry through the drainage catheter and into the cap 106.

Moving to the catheter body 110, as seen in cross section E, the catheter body 110 contains a central lumen 126 formed by the drainage catheter 114. The central lumen 126 is coaxial with the catheter body 110. The catheter body 110 also includes an array of outer lumens 130 formed by outer catheters 132 that are equally dispersed around the central lumen 126 and are adjacent to the outer wall of the catheter body 110. The central lumen 126 and the outer lumens 130 extend through the entire length of the catheter body 110. In this example, there are eight outer lumens 130, which is equal to the number of hollow fiber clusters 122. One cluster 122 of hollow fibers 120 extends into the distal end of each outer lumen 130. In this example, the central lumen 126 is larger than the outer lumens 130. In the example shown, the central lumen 126 has a diameter of between about 0.6 mm and about 1.4 mm, or between about 0.8 mm and about 1.2 mm, such as about 1.0 mm. The outer lumens 130 all have a diameter of between about 0.5 mm and about 1.0 mm, such as about 0.6 to about 0.9 mm, or about 0.7 to about 0.8 mm, such as about 0.75 mm.

The catheter body 110 itself has a diameter of between about 2.7 and about 4.7 mm, or about 3.2 mm to about 4.2 mm, such as about 3.7 mm (11 French) and a length of between about 20 to about 40 cm, such as about 30 cm, for example. The catheter body 110 may be made of an extruded plastic such as PEBAX, or other suitable strong but flexible medical grade material.

At the proximal end of the hollow fiber section 108, the hollow fibers 120 connect to the catheter main body 110. The lumens 126 and 130 may be formed as an extrusion of the catheter body 110. As shown in cross section D, the proximal ends of the hollow fibers 120 are affixed within the outer lumens 130. It can be seen that each of the eight hollow fiber clusters 122 are affixed within one of the eight outer lumens 130 of the catheter body 110. The hollow fibers 120 may be affixed using any method by which the proximal ends of the hollow fibers 120 are securely held in place, forming a seal such that there is no space for direct communication between the outside environment and the outer lumens 130. Further, the open ends proximal of the hollow fibers 120 must not be obstructed when the hollow fibers 120 are affixed inside the outer lumens 130. In cross section E, the empty central lumen 126 and outer lumens are shown as they extend throughout the length of the catheter body 110.

One method of affixing the clusters 122 of hollow fibers 120 in the cap 106 at one end and in the outer lumen 130 at the other end, which may be used in various embodiments, includes first adhering the of hollow fibers 120 of a cluster 122 together at their ends. The hollow fibers 120 may be provided from the manufacturer in clusters which are already adhered together at their ends, or they may be adhered together during manufacture of the catheter 100. An appropriate adhesive may be used to adhere the cluster 122 of hollow fibers 120 together at their ends, such as an epoxy. The ends of the hollow fiber clusters 122 may then be inserted into the cap 106 at one and into the outer lumens 130 at the other end and adhered in the cap and the outer lumens using an appropriate strong and airtight adhesive such as an epoxy. At the same time, the sealed distal and of the drainage catheter 114 may be inserted into the cap and scaled in position between the hollow fibers. A short section of 110 catheter body extrusion may be used to hold and position the clusters 122 and drainage catheter 114.

FIG. 6 shows the proximal end 104 of the catheter 100. The catheter body 110 has a circular cross sectional shape throughout its length. At the proximal end of the catheter body 110, it connects to a manifold 112 which has a flattened, oval cross sectional shape. For example, the manifold dimensions may be about 4 mm to about 6 mm thick by about 12 to about 14 mm wide, such as about 5 mm by about 13 mm, and may be about 20-30 mm long, such as about 25 mm long. The drainage catheter 114 and its central lumen 126 and the outer lumens 130 with the air flow lumens 140, 142 of the catheter body extend into the manifold 112. As shown in cross sections F, G, and H, the central lumen 126 remains in the center of the manifold 112. In contrast, the locations of the outer lumens 130 have shifted from around the circumference of the catheter body 110, as in cross section E, to two groups (a first group 134 and a second group 136) in lateral positions on opposite sides of the central lumen 126, connecting to airflow lumens 140, 142, respectively. As shown in these examples, the first group 134 of outer lumens 130 in airflow lumen 140 ultimately connect to a vacuum, while the second group 136 in airflow lumen 142 ultimately connect to a source of gas, such as dry air, such that the vacuum pulls the gas through, down and back the entire length of catheter 110. Note that the first and second groups 134, 136 and airflow lumens 140 and 142 are identical and interchangeable, such that the first group 134 and first airflow lumen 140 could alternatively be connected to a source of gas and the second group 136 and airflow lumen 142 could be connected to a vacuum. At their proximal ends, the air flow lumens 140, 142 are covered and sealed by plugs 144.

Cross section I shows the drainage catheter 114 extending from the proximal end of the catheter 104. The drainage catheter 114 will be connected to an external drainage line as described later. The external drainage line provides an outlet for bulk fluid drainage from the central lumen 126. After placement of the catheter 100, the plugs 144 will be removed and the air flow lumens 140, 142 will be connected to external catheters which supply gas and vacuum as also described later. The external catheters include a gas supply catheter 204 configured to supply gas to the second group 136 of outer lumens 130 from a gas supply (not shown), and a vacuum catheter 202 configured to apply a vacuum from a vacuum pump (not shown) to the first group 134 of outer lumens 130 (though these connections could be reversed). These catheters 202 and 204 may comprise an air line extension set that connects to a control module.

Cross sections G and H show the section of the manifold 112 in which the outer lumens 130 are funneled into the two airflow lumens 140, 142. The four outer lumens 130 of the first group 134 all connect to the first airflow lumen 140, while the four outer lumens 130 of the second group 126 all connect to the second airflow lumen 142. This connection is provided by a plurality of couplers 138, with one in each outer lumen 130 providing a bridge to the air flow lumens 140, 142. The couplers 138 may be small tubes that are inserted into the lumens proximally and distally and epoxied into place. Alternatively, mandrels and injection molding may be used to create this connection between the outer lumens 130 and airflow lumens 140 and 142. In this way, the manifold 112 provides a connection between the proximal ends of the multiple outer lumens 130 in the main body 110 and the two airflow lumens 140, 142 for connection to the gas supply catheter 204 and the vacuum catheter 202. As described further below, the catheter 100 may be connected to the external catheters 202, 204 and the external drain line via a standard means such as a luer fitting.

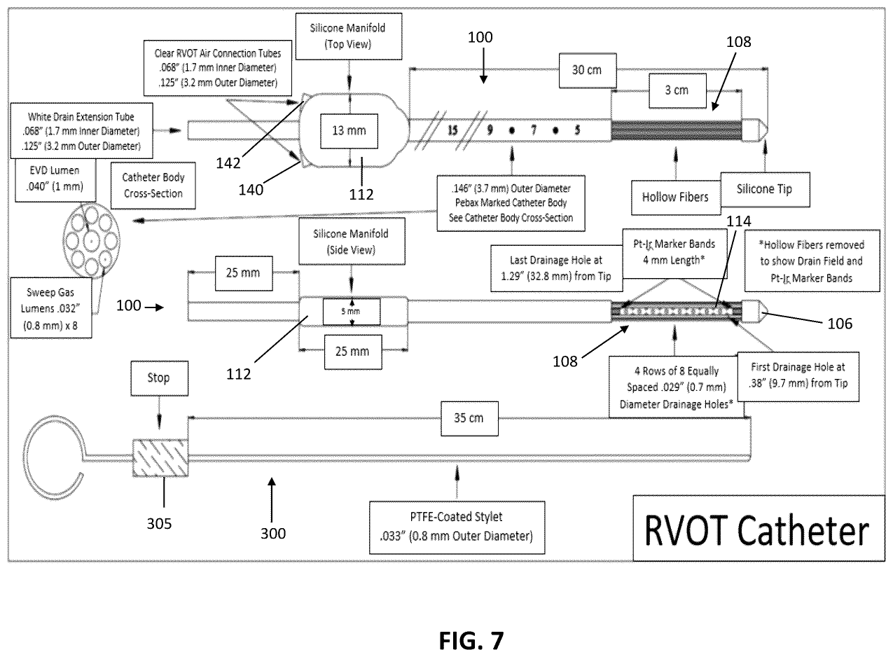

A detailed example of a catheter that may be used in various embodiments is shown in FIG. 7. FIG. 7 shows a top view of the catheter 100 at the top and a side view of the catheter 100 in the middle. The catheter 100 may be inserted into position in the brain with a stylet 300 such as PTFE-coated stylet. When in use, the stylet 300 may be positioned in the central lumen 126 of the catheter. The stylet 300 may be sized to extend through the length of the catheter body 110 to the cap 106, and may include a stop 305 to provide a physical barrier against the distal end of the stylet 305 from contacting or pressing against the cap 106. In this and other embodiments, the catheter 100 may also include markers such as radiopaque markers to assist with placement. In the side view of the catheter 100, a portion of the hollow fibers 120 are cut away for demonstration purposes to show the drainage catheter 114. In this example, the drainage catheter 114 includes four rows of eight equally spaced drainage holes each having a diameter of 0.7 mm, though in alternative embodiments, the drainage catheter 114 could include more or fewer holes, which could be smaller or larger and could be provided in various configurations. In this example, the hollow fiber section 108 further includes Pt—Ir marker bands as radiopaque markers to assist with placement. There are also numerical markers on the exterior surface of the main body 110 to assist the surgeon during placement by indicating depth of insertion of the catheter 100. The dimensions shown in this figure are one example of useful dimensions, but other dimensions may alternatively be used.

The catheter systems may be used with a control module which controls interactions with the catheter and monitors the process of water vapor removal. For example, the catheter module may control the gas flow and the gas vacuum. It may measure pressure within the gas flow lumens, the amount of gas flow, the humidity of the gas after exiting the catheter, the amount of water vapor removed and/or the volume of water removed as water vapor, for example. It may further include safety features to optimize patient safety. It also includes a user interface for communication with an operator. For example, the user interface may be a screen such as a touch screen which displays information (such as monitored functions like pressure and water removal rate) and through which the operator can input operating parameters and other commands to control the operation of the catheter system. The screen may display information graphically and/or numerically.

The control module may be a single, self-contained and enclosed unit within an outer housing (though the functions and components could alternatively be divided into more than one housing.). The control module includes internal components such as one or more processors to store and execute instructions such as software for the operation of the control module, and one or more memory devices for storage of software, patient data, and data acquired during a procedure, for example. The control module may include digital electronic circuitry of various types including integrated circuits, hardware, software, and/or other components in various combinations. It may include machine readable medium such any type of memory which may be used to provide machine instructions and/or data to a processor, which may implement the methods described herein using one or more computer programs. The machine readable medium may store machine instructions in a non-volatile or non-transient manner (such as in a solid state medium) and/or in a transient manner (such as in a cache or random access memory). For example, the control module may include one or more processors for processing and one or more memory components. The processor may be configured to execute programming to perform the various steps described herein. The memory may store data such as patient data input by an operator. The memory may further store programs such as an operating system and one or more application programs which operate on the processor. The control module may further have components for transmission of data, such as a data port like a USB connection and/or other data connection, or components for wireless connection such as blue tooth, wifi, and/or other data transmission components. The control module may connect to a source of power through a plug for connection to a power cord, or may include a power cord for connection to a power source, and may optionally include an internal battery such as a rechargable battery for continued power supply in the event of a loss of external power.

The control module may further include an unrestricted vent for establishing a zero-pressure inlet reservoir of gas from which gas is supplied to the gas supply catheter. The zero-pressure reservoir may be located in the control module, in line between the gas supply source and the gas supply line to the catheter. The use of a zero-pressure reservoir provides patient safety, in that the gas supplied to the catheter is never under pressure so there is no risk of gas passing from the hollow fibers into the ventricle. The control module may further include a vacuum generator for regulating the gas flow from the zero-pressure reservoir to the gas supply catheter.

In various embodiments, the control module includes a plurality of ports on the outer housing. For example, the control module includes a primary gas supply inlet port. The primary gas inlet port connects to an external gas source, such as a dry air source such as medical air or other non-medical filtered air, or a mixture of air and another gas such as a mixture of air and carbon dioxide such as 95% air and 5% carbon dioxide. The control module may also include a vacuum outlet port for connection to a vacuum supply catheter to the catheter 100 and a gas supply outlet port for connection to a gas supply catheter to the catheter 100. In some embodiments, the vacuum outlet port and the gas supply outlet port may be combined, such as in a gas inlet/outlet port, which may be a dual chambered pneumatic port that communicates with the catheter 100 via the gas supply catheter and the vacuum catheter.

The control module may further include a plurality of controls such as pneumatic controls for purging the gas supply catheter and the vacuum catheter with dry gas and turning the operation on and off. The purge function may be used to clear the internal air lines of the controller from moist air or condensate. The control module may include valves that shut off airflow to the catheter during purging such that purging does not affect the catheter. Purging can occur, for example, when starting RVOT, and it can be done on a scheduled basis to purge moist air depending on RVOT intensity level and/or intake air humidity levels.

The control module may include a plurality of sensors. For example, the control module may further include one or more gas flowmeters for continuously monitoring the flow of gas during an RVOT procedure. In one embodiment, the gas flow meter is located just before the RVOT catheter inlet tec. FIG. 8 shows a diagram of an example of components and connections for the RVOT pneumatics of the airflow within the control module.

The control module may further include a humidity sensor for continuously monitoring the water content of the gas exiting through the vacuum catheter during an RVOT procedure. The control module may further include a temperature sensor for continuously monitoring the temperature of the gas supplied to the gas supply catheter. In various embodiments, the control module may include one or sensors to measure humidity and/or temperature of the sweep gas upon exiting the control module as the gas is supplied to the catheter 100. The control module could alternatively or additionally include one or more sensors to measure the humidity and/or temperature of the intake gas, such as if ambient, filtered air is used as an alternative to compressed medical dry air. The control module may further include one or more pressure sensors, such as an outlet gas pressure sensor for verification of the vacuum level of the gas after exiting the vacuum supply catheter to supply the catheter 100.

The control module may include a humidity sensor to measure humidity of the sweep air after exiting the catheter. These humidity measurements may be used by the control module to calculate and track the volume of water removed from the CSF in the sweep gas. This data may be stored by the control module and displayed and/or supplied to an RVOT operator.

The amount of water removed may be determined by the Water Removal Rate (WRR) times the amount of time the RVOT function is on and set at an intensity level such as one of four RVOT intensity settings (12.5%, 25%, 50%, or 100%). The RVOT Controller may calculate the WRR based on the parameters measured within the Controller and the RVOT intensity setting. The WRR may be calculated as follows:

$$WRR ( g / hr ) = 0.06 RVOT Intensity \text{} ( ON / ON + OFF ) Flow ( LPM ) Corrected Humidity$$

The Corrected Humidity (CH) may be calculated as follows:

$$C H = [ \frac{760}{273.15} \times \frac{Humidity ( \frac{g}{m 3} ) ( 273.15 + T ( C ) )}{760 + VP ( mmHg )} ] - Purge Gas Humidity$$

Purge Gas Humidity may be calculated during each purge cycle for WRR calculations until the next purge cycle. The Purge Gas Humidity may be calculated as follows:

$$Purge Gas Humidity = \frac{760}{273.15} \times \frac{HumidityPurge ( \frac{g}{m 3} ) ( 273.15 + T P u r g e ( C ) )}{760 + VPPurge ( mmHg )}$$

During intermittent operation the WRR may be calculated and displayed on the controller, such as at the end of each “ON” cycle, to represent the average WRR over the entire cycle time (such as 4 or 8 minutes). The Purge Gas Humidity parameter may be compared to the Humidity Dry Threshold, such as at the end of each purge cycle, which may be set in the in the controller functions, such as in the Configuration Settings Parameter Order and Range screen.

The control module may include a variety of other safety features, many of which relate to ensuring that there is never any pressure in the gas supplied to the catheter, that is, that the gas supplied is at atmospheric pressure. There may be multiple layers of safety measures to ensure this is always true. For example, it may include a pressure regulator to down-regulate gas pressure at the primary gas inlet. It may further include a gas pressure sensor to detect gas pressure in the primary gas inlet line and may alert an operator to the presence of either over-pressure or under-pressure situations. If the gas supplied to the control module, the control module may include one or more pressure reduction stages. For example, if medical air is used as dry gas, it may be pressurized initially, such as at about 2200 psi, and this needs to be reduced to zero (gauge) pressure. To accomplish this, the control module may include a first stage pressure reduction such as a pressure regulator which may reduce the pressure of the supplied gas to about 0 to about 25 psi. The gas may then flow to a second stage which is an unrestricted vent to ensure the gas is reduced to 0 psi. In addition to the pressure regulator at the primary gas inlet, it may include a pressure relief valve downstream of the primary gas inlet in case of an occurrence of an over pressure in which pressure is above a threshold, in which the gas is released to the outside environment.

As mentioned above, the control module may include a zero pressure reservoir of the supplied gas. The reservoir may include a large exit port, which may have an unrestricted vent, such that the zero pressure reservoir is maintained at atmospheric pressure, further preventing any overpressure of the gas supplied to the catheter.

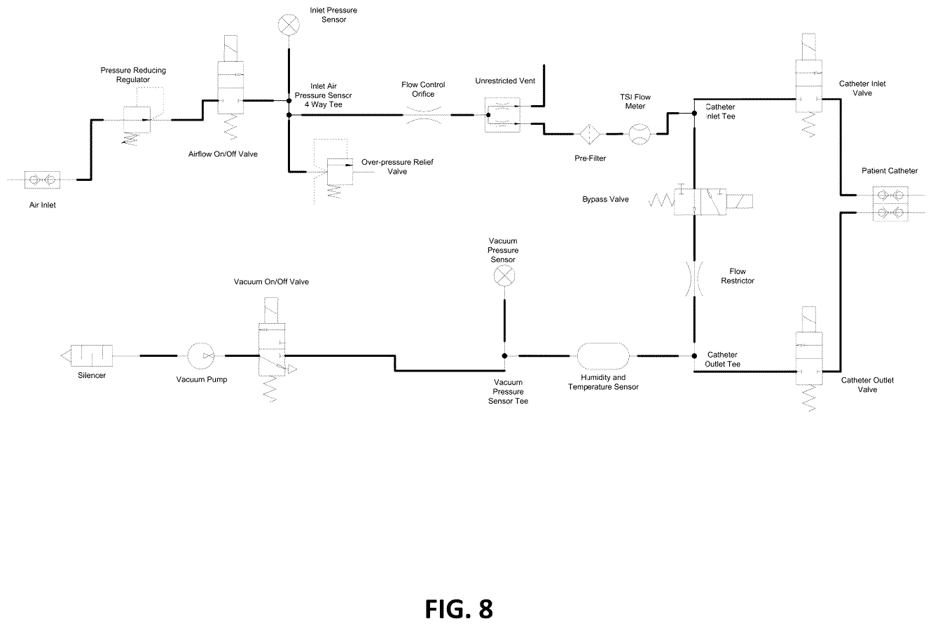

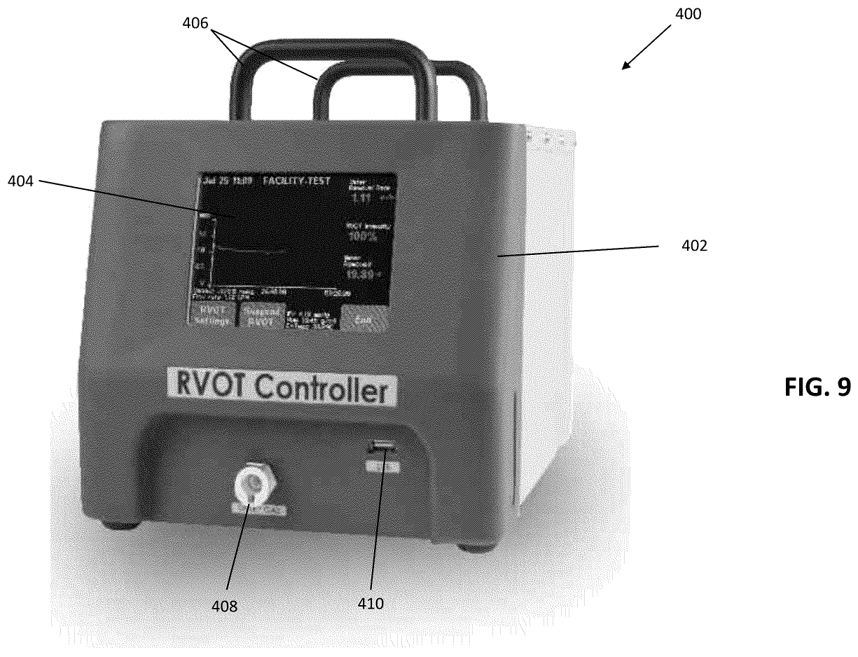

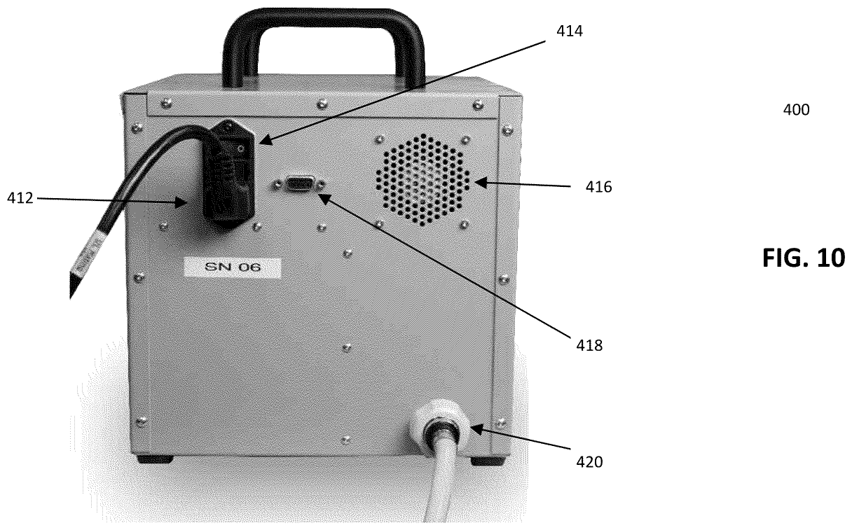

An example of a control module 400 that may be used in various embodiments is shown in FIGS. 9 and 10, which show a front and back view of the control module 400. The control module includes an outer housing 402 which surrounds the control module and carrying handles 406. The front of the control module includes a touchscreen display 404 for user input and monitoring of device operating parameters, an air supply port 408, and a data port 410. In this example the air supply port 408 is a twin tube female connector for connection to the air extension line set which includes the gas supply catheter 202 and the vacuum catheter 204 for connection to the catheter. As such, the air supply port 408 includes both an air supply port and a vacuum connection. In alternative embodiments, the air supply connection and the vacuum connection could be provided as separate ports. In this example, the data port 410 is a USB connector and may be used for the upload of patient data to the control module 400. The back of the control module 400 includes a power supply cord 412, an on/off switch 414, an exterior air vent 416, another data port 418, and a gas inlet port 420 for connection to a gas supply source. The locations of these various connections and components may be other than as shown, and may include other components in addition to or in the alternative to those shown.

The control module may provide a continuous steady flow of dry sweep gas through the catheter during the RVOT procedure. In such embodiments, the removal of water may be controlled by increasing or decreasing the airflow by increasing or decreasing the vacuum level and/or through the use of airflow resistors in the control module. However, such controls may lack accuracy. Therefore, in some embodiments, it may be preferable to provide the sweep gas intermittently, in a repeated on and off cycle throughout the RVOT procedure in order to more accurately control and optimize the removal of fluid. For example, the control module may be programmable to provide full airflow intermittently, as input and directed by an operator, to control the rate of fluid removal.

In general, when intermittent (on and off cycles) of airflow are used for RVOT therapy, it may be desirable to minimize time between cycles (ON/OFF) in order to maximize the long term osmotic effect. A formula for calculating osmolality at any time point is presented below, and this formula will allow estimation of the most efficacious use of ON/OFF cycles, but other factors may play a role. For example, very short On or Off cycles may not closely follow the mathematical model due to ‘inertia’ in the biological system or the first few seconds of closing or clearing airflow lines. Longer Off cycles raise concerns about development of condensate within the hollow fiber lumens, and formation of liquid water with its increased resistance to flow, and condensation is possible due to local evaporative cooling in adjacent hollow fibers. Very long Off cycles could result in return to baseline, so there is no actual increase in CSF osmolality over time. These factors could decrease the accuracy of the control of the water removal rate. Therefore, when an RVOT procedure is performed using an on and off cycle, the durations of both the on period and the off period must be carefully determined.

The ideal duration of the on and off period may depend upon the specific design parameters of the RVOT catheter as well as the amount of vacuum and the rate of gas flow. However, when an RVOT catheter like the ones described herein is used, the following on and off cycles may be used to optimize control over the water removal during the procedure. For 100% maximum water removal rate, the air flow may be on continuously. For 75% of the maximum water removal rate, the airflow may be cycled on for about 3 minutes and off for about 1 minute. For 50% of the maximum water removal rate, the airflow may be cycled on for about 2 minutes and off for about 2 minutes. For 25% of the maximum water removal rate, the air flow may be cycled on for about 2 minutes and off for about 6 minutes. For 12.5% of the maximum water removal rate, the air flow may be cycled on for about 1 minute and off for about 7 minutes. Further examples and explanations are provided in the Examples section at the end of this disclosure.

In use, the catheter may be used with various other components which may be included in the catheter system. For example, the catheter system may include a trocar, such as a trocar with a protective sheath, for tunneling and placement of the catheter. It may also include a CSF Luer fitting with a plug for the drain catheter 114. It may further include the gas supply catheter and the vacuum catheter, for connecting the control module to the catheter. The gas supply catheter and/or the vacuum catheter may include anti-bacterial inline filters and connections.

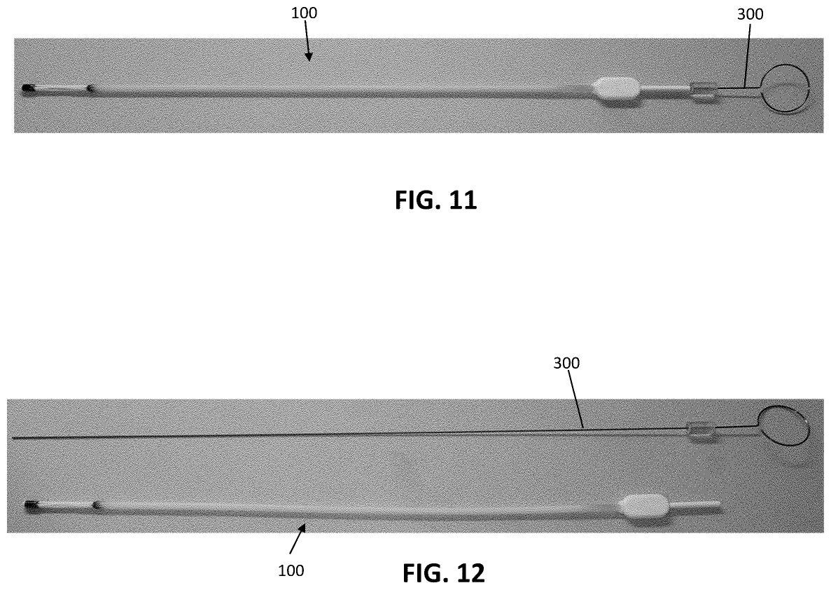

In use, the catheter may be positioned with the hollow fiber section of the catheter in the lateral ventral through a placement process including the use of a trocar. After preparing the surgical site with a craniotomy, the distal end of the catheter is first inserted through the craniotomy hole, through the brain tissue and into the lateral ventricle, with the proximal end of the catheter remaining outside the skull. Because the catheter is flexible, this process is performed with a stylet in place inside the central lumen of the catheter to provide the necessary stiffness as shown in FIG. 11. The positioning of the catheter may be monitored and assessed through external markers on the outer surface of the catheter and radiographically through Xray imaging to see the location of the radiopaque markers of the hollow fiber section and/or other radiographic marker. The stylet is then removed, as shown in FIG. 12 (although the catheter will, at this point, be positioned in the brain). At this point, the catheter 100 is in position and ready to use. The proximal end of the catheter 100 may be connected to the air supply and vacuum catheters and optionally to a drain catheter for bulk fluid removal (or these lines may have been attached previously). However, it may be preferable to further position the proximal end 104 of the catheter 100 away from the craniotomy hole. This distancing of the exit point of the catheter 100, away from the craniotomy hole, may reduce risk of infection while the catheter remains in place. This may be done by tunnelling the proximal end 104 of the catheter 100, including the manifold 112, away from the craniotomy hole (and the underlying entry point of the catheter 100 into the brain), as described further below.

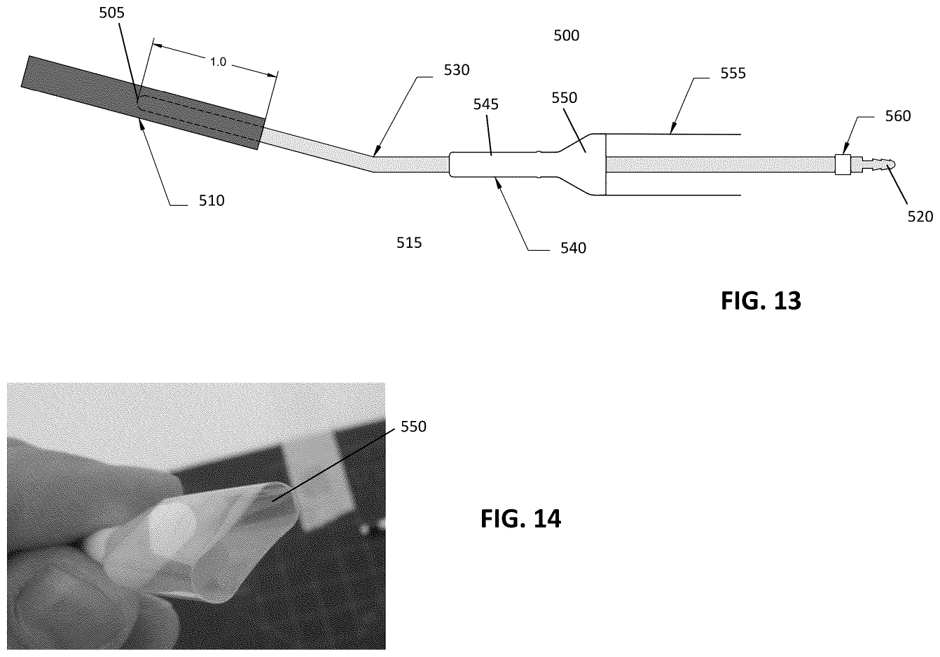

Once the distal end 106 of the catheter 100 is in position and the stylet 300 has been removed from the catheter 100, the proximal end 104 of the catheter 100 may be connected to a trocar such as the trocar 500 shown in FIG. 13. The trocar includes a tip 505 which is used for tunneling. In this example, the tip 505 is pointed but it may alternatively be rounded. A protective cover 510 over the tip 505 is removed prior to use. The main body 515 of the trocar is a rigid and elongated shaft, constructed of a strong material such as metal and extends from the tip 505 at one end to the connector 520 at the opposite end. The connector 520 is configured to connect to the proximal end of the catheter 100, specifically to the drainage tubing 114. In this example, the connector 520 is a series of three tapered barbs, though other number or shapes of barbs or other features could be included at the end to facilitate connection to the drainage catheter. The main body 515 is straight, except for one soft angle 530 at which it bends as shown. The angle could alternatively be larger or smaller, or the main body 515 could be straight with no bend, or it could be curved, or any preferred shape for assisting with the tunnelling procedure. In the embodiment shown, the small angle assists with tunnelling the tip under the scalp a short distance and then turning the trocar to angle the tip outward to exit.

The trocar 500 includes a sheath 540 which is surrounds the main body 515 and is slidable on the main body 515. The sheath 540 includes a neck portion 545 that has a circular cross-sectional shape and abuts the main body 515 and is oriented toward the tip 505 of the trocar 515. Behind the neck portion 545 is a shoulder portion 550 of the sheath 540 which flares outward at an angle of approximately 45 degrees relative to the main body 515 and neck portion 545,as it extends from the neck portion 545, though a larger or smaller angle is possible. For example, the shoulder portion 545 could be at an angle of between approximately 20 and approximately 75 degrees, or between approximately 30 and approximately 60 degrees, or between approximately 40 and approximately 50 degrees. Note, however, that the shoulder portion 545 flares outward in only one horizontal plane. In the other plane, perpendicular to the plane in which the shoulder portion 550 flares outward, the shoulder portion 550 does not flare outward but rather remains approximately the same thickness as the neck portion and the sleeve portion 555 (described further below). As such, the shoulder portion 550 has an oval cross section shape, with an oval that increases to widen in the long dimension as it progresses away from the neck 545. The shape is therefore flared in one dimension but flat and uniform in the other, perpendicular dimension.

Extending from the shoulder portion 550, toward the connector 520 end of the trocar 100, is a tubular sleeve portion 555 of the sheath 540. Like the shoulder portion 550, the sleeve portion 555 has an oval cross sectional shape, though unlike the shoulder portion 550, it is consistent in shape and size throughout its length. The sleeve portion 555 surrounds the main body 515 of the trocar 500 and is spaced away from the main body 500 with a gap between the main body 500 and the sleeve portion 555 in the long cross sectional dimension. The sleeve portion 555 retains approximately the same cross section shape throughout. The sleeve portion 555 is therefore uniformly wider in one cross sectional dimension than the other. Indeed, as described further below, the sleeve portion 555 is sized to accommodate the wide and flat manifold 112 of the catheter 100 proximal end 104.

When seen from the perspective shown in FIG. 12, which is referred to herein as a top view, the shoulder portion 550 flares outward and the sleeve portion 555 is wide and spaced apart from the trocar main body 515 which extends through the center. However, in a side view, at 90 degrees from the top view, the shoulder portion 550 would not appear to flare, and the sleeve portion 555 would not appear to be spaced apart from the main body 515. Rather, the entire sheath 540 would appear to have an equal width through its length. This may be appreciated, in part, from the photo of the sleeve portion 555 shown in FIG. 13, in which the oval shape of the end of the sleeve portion can be seen.

The neck portion 545, shoulder portion 550, and sleeve portion 550 of the sheath 540 may be constructed of the same material, and as such may be different portions of a single piece, or they may be constructed from different materials which are affixed together to form a single piece. In either case, the exterior surfaces of the portions connect smoothly, which no breaks or ledges, to allow the sheath 540 to pass smoothly through the tissue.

The sheath 540 is slidable along the main body 515, which allows for easier connection of the trocar 500 to the catheter 100 with subsequent covering of the manifold 112. In the embodiment shown, the sheath 540 is prevented from sliding forward past the angle 530 by the presence of the angle 530. The trocar 500 also includes a stop 560 which encircles the main body 515 near the connector 520 and prevents the sheath 540 from sliding past the stop 560 and sliding off the trocar 500. As the trocar 500 is advanced through the tissue, the tissue may resist passage of the flared shoulder portion 550 of the sheath 540 and may create a force pushing back against the shoulder portion 550. This force is transmitted to the stopper 560 which prevents the sheath 540 from sliding off the trocar 500. This force transmission to the stopper 560, rather than to the attached catheter manifold 112 within the sheath 540, prevents force against the manifold 212 from pulling the catheter 100 and disconnecting it from the trocar 500. In addition, as will be discussed further below, the presence of the sheath 540, surrounding the manifold 112 and the airflow lumens 140, 142, protects the airflow lumens 140 142. In this way, the sheath 540 prevents the plugs 144 from dislodging from the airflow lumens 140, 142, keeping the lumens clean and keeping tissue out of the lumens even while the catheter proximal end 104 is pulled through the tissue.

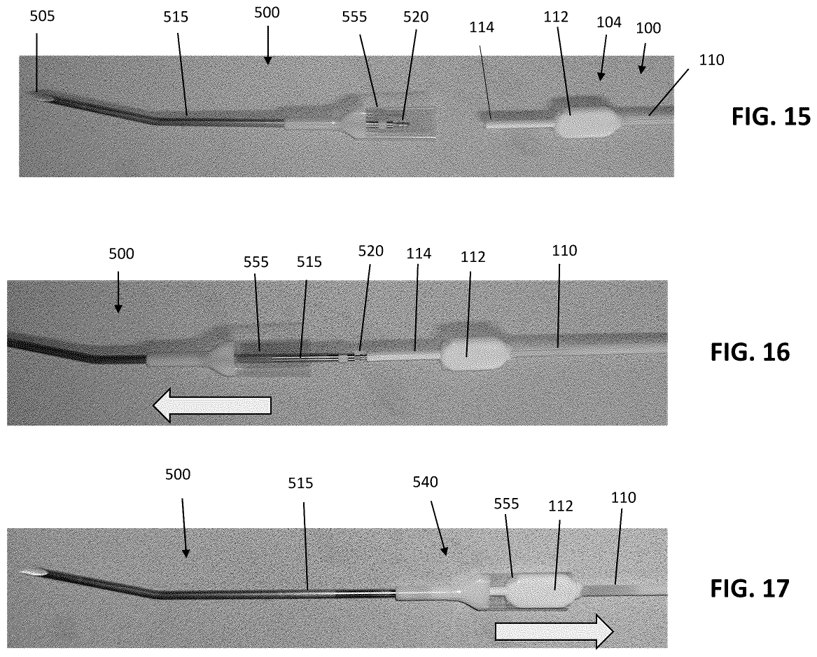

The proximal end 104 of the catheter 100 may be connected to the connector 520 end of the trocar 500 as shown in FIGS. 15-17. The connector 520 at the end of the trocar 500 is sized to fit snugly inside of the drainage catheter 114 at the proximal end 104 of the catheter 100, where it is held inside the drainage catheter 114 by tapered barbs which are shaped to enable insertion into the open proximal end of the drainage catheter 114 but which will resist removal from the drainage catheter 114. In FIG. 15, the trocar 500 and catheter 100 are separate, and the sheath 540 covers the connector 520 of the trocar end. Next, in FIG. 16, the sheath has been slid back as indicated by the arrow to expose the connector 520, and the connector 520 is partially inserted into the open proximal end of the drainage catheter 114. Then, in FIG. 17, the connector 520 fully inside the lumen of the drainage catheter 114 such that the trocar 500 and the catheter 100 are securely connected. The sheath 540 is then slid over the point of connection between the connector 520 and the drainage catheter 114 as indicated by the arrow. In addition, the sleeve portion 555 surrounds nearly all of the manifold 112. In alternative embodiments, the sleeve portion 555 could cover between half and all of the manifold 112. In any case, the sleeve portion 555 will cover and surround at least the portion of the manifold 112 including the air supply and vacuum supply lumens 140 142 to prevent dislodging of the plugs from lumens during the tunneling procedure, which could result in tissue or fluid entering the lumens.

Because of the wider pathway required to fit the sheath 540 and the manifold 112 within the sheath 540 through the tissue, it may be desirable to create a pathway through the tissue before inserting and tunnelling the trocar 500. Therefore, in some embodiments, before using the trocar 500 to tunnel through the tissue, the surgeon may first create a tunnel as a pathway between the scalp and the skull using an appropriate tool such as a hemostat. In this way, a wider tunnel may be created before using the trocar 500, to accommodate the wider sheath 540 of the trocar 500 and the catheter manifold 112 within it, prior to passage of the trocar 500 and catheter 100. The tunnel may extend from the craniotomy hole, beneath the scalp a short distance such as about 2 to about 4 centimeters, or another distance as desired, to then exit the body through a small incision in the scalp. When a tunnel is prepared prior to passage of the trocar 500 through the tunnel, a trocar 500 with a rounded or blunt tip may be used, though it may alternatively be sharp. However, if the tunnel is not created first and the trocar 500 is used to form the tunnel, then embodiments of the trocar 500 having a sharp tip may be used.

Once the tunnel has been prepared and the catheter 100 attached to the trocar 500, the trocar 500 can be passed through the tunnel from the craniotomy location to the scalp exit location and passed out through the scalp at the exit, pulling the proximal end 104 of the catheter 100 through the tunnel in a retrograde fashion. Likewise, if the trocar 500 is used to create the tunnel, the trocar 500 is passed between the skull and the scalp, beginning at the location of the craniotomy to form a tunnel. The trocar 500 then exits through the scalp at a location away from the craniotomy, thereby forming a tunnel, with additional force required to create the tunnel and to pull the trocar 500 and proximal end 104 of the catheter 100 through the tunnel.

Once the proximal end 104 of the catheter 100 has been pulled through the tunnel and exits the body, it is disconnected from the trocar 500. This can be done by pulling the drain catheter 114 off the connector 520, though the connection is ideally strong which may make this difficult. Alternatively, the drain catheter 114 may be cut between the manifold 112 and the trocar connector 520 to separate the catheter 100 from the trocar 500. The disconnected end or freshly cut end of the drain catheter 114 can then be used for connection to a CSF drainage system, such as through connection such as a Luer connection, if desired.

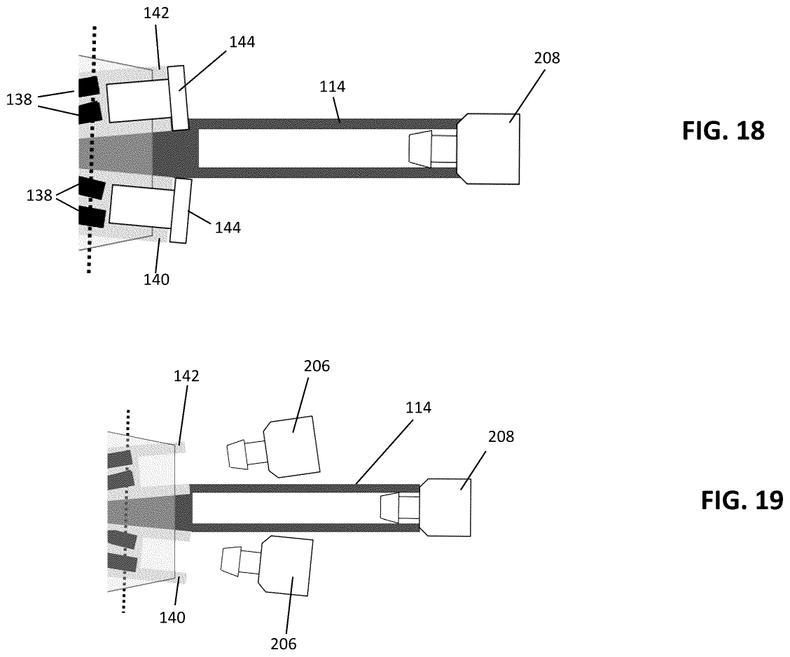

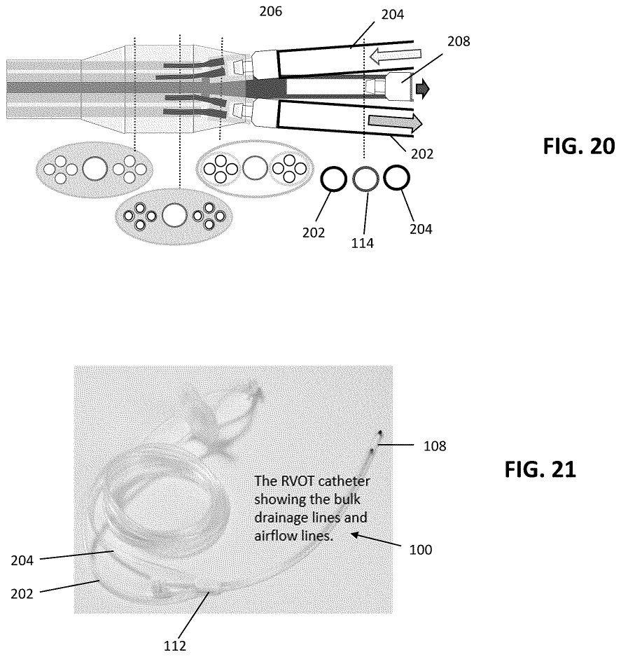

FIGS. 18-20 show the process of connection of the proximal end of the catheter 100 to external lines after placement. In FIG. 18, the drain catheter 114 has been cut to disengage the trocar 500 and a Luer connection 208 has been placed in the drain catheter. The Luer connection may connect the drain catheter to a CSF drainage system. The plugs 144 are present in the airflow lumens 140, 142. Next, as shown in FIG. 19, the plugs 144 are removed from the airflow lumens 140, 142 and Luer connections 206 are inserted in their place. Finally, in FIG. 20, the airflow lines (gas supply and vacuum supply lines 204, 202) are connected to the Luer connections 206. The proximal ends of the airflow lines are connected to the control module (not shown).

An example of a catheter 100 connected to airflow lines is shown in the photograph shown in FIG. 21. The gas supply and vacuum supply lines 204, 202 connect to the catheter 100 at the proximal end of the manifold 112. The drain catheter 114 is connected to a Luer connector 208.

As can be appreciated, in order to tunnel beneath the scalp, the proximal end 104 of the catheter needs to be small. However, the connection between the outer lumens 124 into the air supply and vacuum supply lumens 140 and 142 necessarily increases the required volume. By moving these connections laterally, on each side of the central lumen 126, the catheter 100 dimensions expand in only one direction and remain smaller in the other direction, taking on an oval cross section at the manifold 112 which is flat and therefore easier to pass through the tissue.

After placement of the catheter 100 is complete, the hollow fiber section 108 are in the lateral ventricle and the proximal end 106 of the catheter 100 spaced away from the craniotomy location and extends outside of the patient's body. Where the catheter 100 exits the skull through the craniotomy hole, the main body 110 makes a sharp turn and must bend approximately 90 degrees into the tunnel. The lumens within the main body 110 must remain open to unobstructed airflow for the catheter 100 to function properly. The use of the outer lumens 130, which are separated by and surrounded by the solid main body 110 material, resist compression even when turning at a right angle. That is, rather than having a pair of single lumens extending through the length of the main body (one to supply gas and the other to supply vacuum) to the hollow fibers, the multiple lumens extend through the main body, with a first plurality of lumens such as 4 providing gas and a second plurality of lumens such as 4 providing vacuum. These separate lumens perform well while maintaining a small diameter catheter and they resist compression even when bent upon exiting the cranial cavity. It is further believed that separating the gas supply and the vacuum supply into multiple lumens in the main body of the catheter may maintain a more even supply of gas to all of the hollow fibers. In addition, as compared to having the hollow fibers extend through the main body, it is believed that the use of the outer lumens provides reduced resistance and therefore better gas flow.

### EXAMPLES

To better compare different combinations of ON/OFF cycles and optimize water removal, a mathematical model can be used. The formula below was used in the following examples to predict osmolality at any time point (OSM (t)) using the variables of CSF production rate (Q in), ventricular volume (VOL), and RVOT Water Removal Rate (WRR). The expected target osmolality (OSMtarget) can be modeled based on bench studies, the formulas, or mass balance equations, which all give similar results. For the examples below, the target change in osmolality is 32 mosm at 100% RVOT Intensity.

$$OSM ( t ) = ( {\underline{OSM}}_{\underline{target}} - {\underline{OSM}}_{\underline{starting}} ) \times ( 1 - {e}^{- ( {Q}_{in} - WRR ) t / VOL} )$$

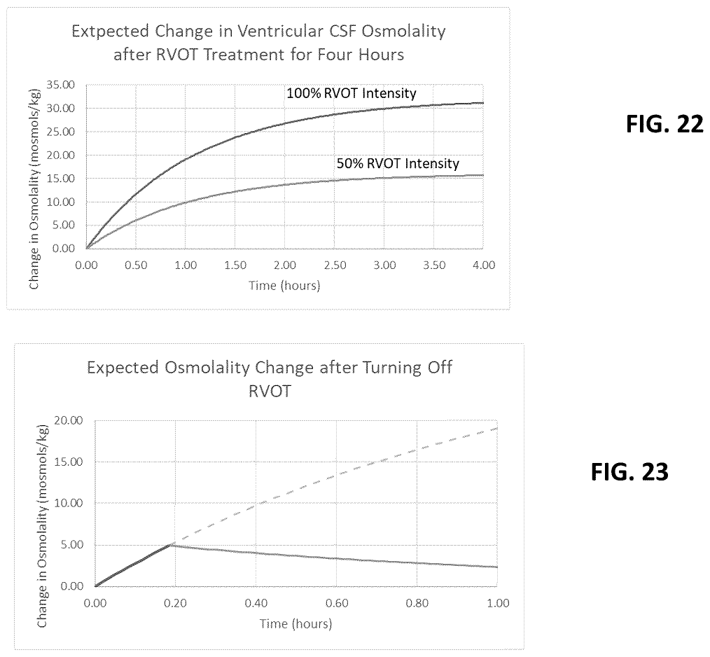

A simulation was performed using the parameters mentioned above with a CSF production rate of 10 ml/hr and a ventricular volume of 10 mls, and a water removal rate of one ml/hr and with 100% RVOT intensity and with 50% RVOT intensity. The expected change osmolality at any time point of the first four hours are shown in FIG. 22.

As mentioned above, however, the mechanics of achieving a 50% Intensity Level in water removal requires a more complicated analysis due to the use of ON/OFF periods, and the different rates of change. (OSM_{target}−OSM_{starting}) during the RVOT ON period is still a differential of 32 mosm. When turning RVOT OFF after reaching an increase of 5 mosm, the (OSM_{target}−OSM_{starting}) term now is −5 mosm, so the rate of falling osmolality while OFF is about 15% of the rate of increase with RVOT ON. FIG. 23 demonstrates this graphically as the osmolality rises rapidly when turning ON RVOT, modeling the rise over the first 12 minutes given the expected asymptote of (delta) 32 mosm. With this exponential function, the osmolality rises quickly in the early minutes. When the RVOT is turned OFF, however, the osmolality drifts back down, but the rate of drift down is much lower than the rate of increase because there is only a 5 mosm difference between the starting level and the expected target of returning to baseline osmolality. If the RVOT was turned on again at any point along that decay, osmolality would start to rise quickly again. The ON setting is always at maximum vacuum, so the Δmosm is always 32.

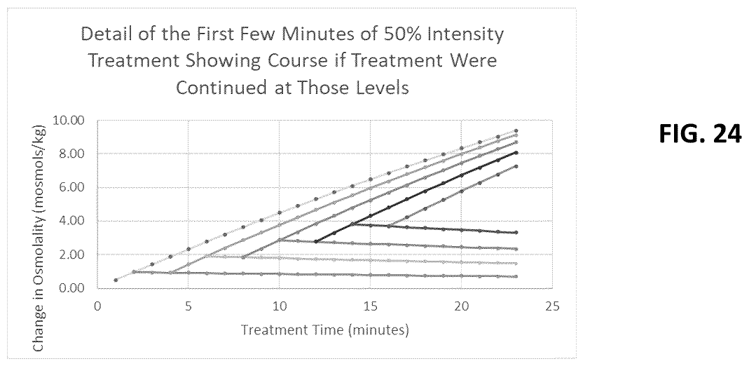

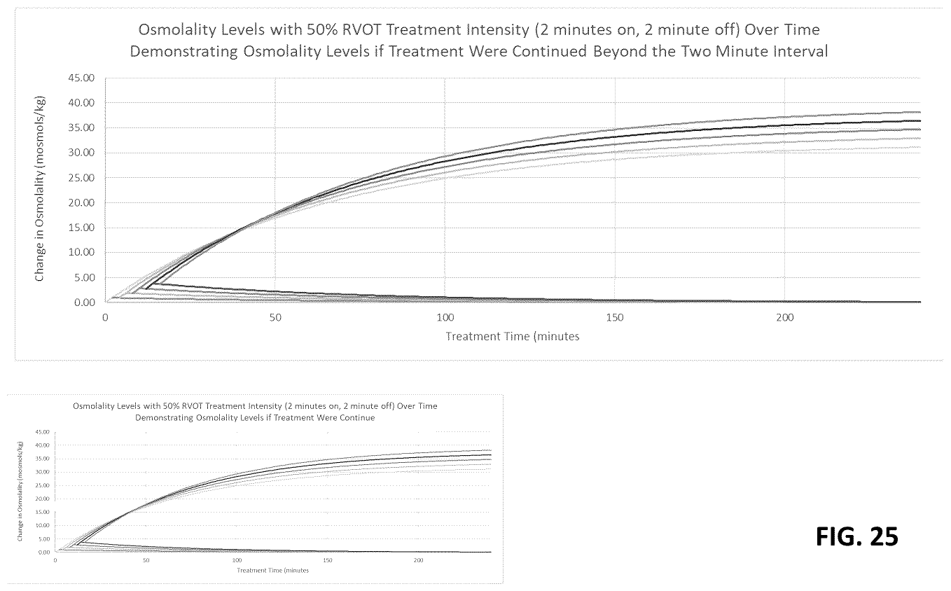

In another example, a model was run of a 50% intensity RVOT treatment using a treatment cycle of two minutes on and two minutes off. The results for the first several minutes are shown in FIG. 24 Each tracing is presented as if it had never been turned off after each new two minute starting point. This allows the visualization of how the exponential function would rise or decay over time. FIG. 25 expands this view, showing the final asymptote of each start/stop time over several hours. would while the results for the first several hours are shown in FIG. 25. This example demonstrates the concept that, no matter what the starting point of treatment during a treatment period, the increase in osmolality during the ON setting should be the same, which in this case is delta 32 mosm. Note that each asymptote is slightly higher as the ON-OFF cycles are repeated if the last ON cycle was allowed to remain on. This is explained by the stepped up initial osmolality at each ON cycle. The initial osmolality rises roughly 2 mosm for each on cycle, and falls very slowly during the off cycles, so OSM_{starting} is 0, 2, 4, 6 mosms, etc, but the target mosmols/kg (if the on cycle were left on), would be Δ32, or OSM_{target} at 32, 34, 36, 38 mosms, and so forth. For the off periods, however, the expected the target would be zero, or a return to the untreated state. Thus, there is a slower decay but the rate of decay increases with each ON-OFF cycle (with OSMstarting at progressively higher levels). Eventually an equilibrium will be attained in which the drop in osmolality over the two minute on period is equal to the increase in osmolality over two minute off period, and this equilibrium can be maintained steadily over several hours.

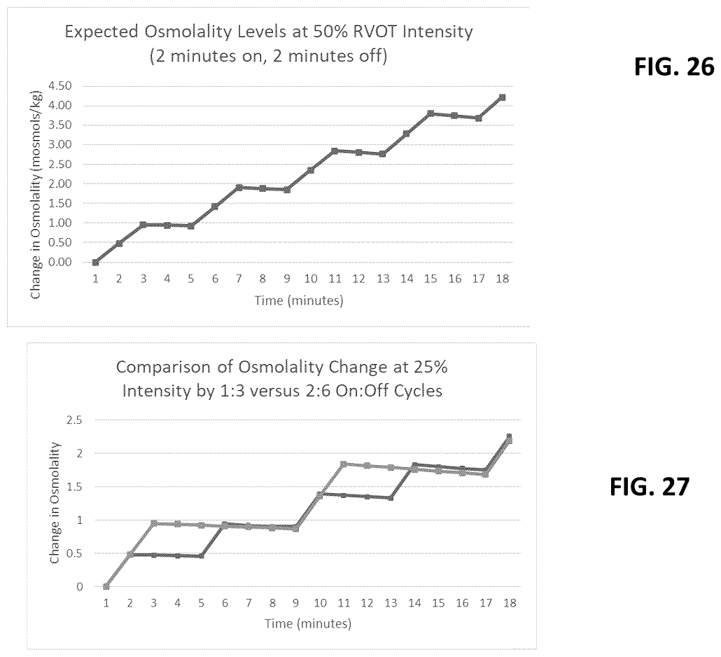

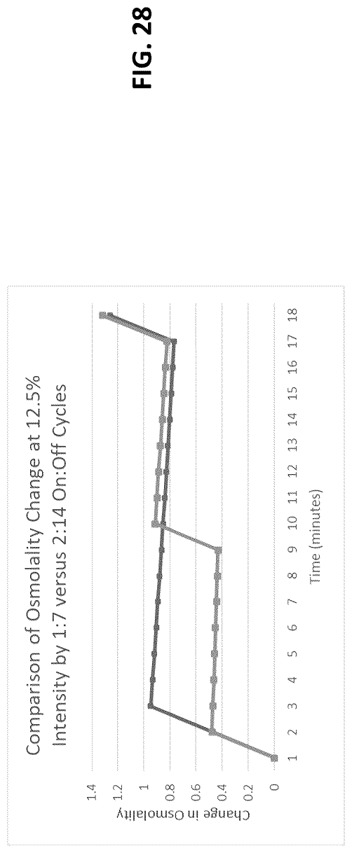

The actual expected change in osmolarity with treatment is shown in the FIG. 26. FIG. 26 shows the predicted osmolality level when RVOT is turned on to maximum flow for two minutes and then turned off for two minutes and repeated several times. The change osmolality is a function of water removal as well as the other factors noted above. The osmotic gradient produced by this change can be quantified by calculating the area under the curve. The area under the curve can then be used to compare durations of on and off cycles over different time periods.

In another example, two options were compared to achieve 25% RVOT intensity with ratio of time on to time off of 1 to 3 for a total of four cycles. In one option, the on/off cycle was 1 minute on and 3 minutes off. In the other option, the on/off cycles was 2 minutes on and 6 minutes off. The results are shown in FIG. 27. The area under the curve was calculated for both options. Comparing the areas under the curves for the two options demonstrates that the 2 to 6 produces a greater gradient at least in this early time. The area under the curve for the 2 minute on/6 minutes off cycles was 11.4% greater than the 1 minute on/3 minute off cycle. Therefore, the 2 minute on and 6 minute off cycle would be selected for use in RVOT therapy.

In a further example, models of different on/off cycle durations were compared at 12.5%, which may be the lowest intensity in various embodiments. Two immediate options were compares: 1 minute on/7 minutes off; and 2 minutes on/14 minutes off. Using the model and calculating the area under the curve, the cycle of 2 minutes on and 14 minutes off produced the greatest area under the curve, and as such would seem to be preferred for producing a change in osmolality. However, there is competing concern. The presence of no airflow for too long a period could result in variation in airflow through the hollow fibers due to condensation. As noted above, the high humidity can lead to an increase in airflow resistance with development of fluid within the fibers. This can particularly occur due to the evaporative cooling in adjacent fibers. Therefore, the ratio of 1 to 7 may be selected to avoid the longer static period.

The embodiments described herein primarily contemplate the use of the hollow fibers for water removal from the lateral ventricles of the brain. However, the catheter system could alternatively be used for water removal from the CSF in other locations of the brain or spinal cord, or to remove water from other areas of the body.

In the foregoing description, the inventions have been described with reference to specific embodiments. However, it may be understood that various modifications and changes may be made without departing from the scopes of the inventions.

The invention claimed is:

1. A catheter system for reduction of cerebral edema comprising a catheter, the catheter comprising:
an elongated main body comprising a proximal end, a distal end, a central lumen and a plurality of outer lumens, the central lumen and the outer lumens extending therethrough and the outer lumens positioned around the central lumen;
a hollow fiber portion distal to the main body, the hollow fiber portion comprising
a plurality of hollow fibers comprising elongated hollow tubes with semipermeable walls, the semipermeable walls being gas permeable and liquid impermeable, the hollow fibers each comprising a proximal end connected to one of the plurality of outer lumens, and
a drainage catheter, and the drainage catheter comprising a central lumen, a plurality of pores, the drainage catheter having a proximal end connected to the central lumen of the elongated main body and a distal sealed end, the plurality of hollow fibers are positioned around the drainage catheter;
a cap distal to and connected to the hollow fibers, the cap comprising an outer wall that forms a central space into which the hollow fibers extend and terminate at open distal ends, the central space forming a plenum for gas sweeping through the outer lumens and the hollow fibers.

2. The catheter system of claim 1, the catheter further comprising a manifold proximal to the main body, wherein the outer lumens split into a first group and a second group lateral to the central lumen.

3. The catheter system of claim 2 wherein the manifold has an oval cross sectional shape.

4. The catheter system of claim 1, the catheter further comprising a drainage catheter extension extending proximally from the manifold.

5. The catheter system of claim 4 further comprising a trocar comprising an elongated main body, a sheath, and a connector end, wherein the connector end is configured to connect to the drainage catheter extension of the catheter.

6. The catheter system of claim 5 wherein the sheath is slidably engaged with the trocar main body and configured to slide over and around the manifold of the catheter when the catheter and the trocar are connected.

7. The catheter system of claim 1 wherein the hollow fibers have an outer diameter between about 100 and about 500 micrometers.

8. The catheter system of claim 1 wherein the semipermeable walls of the hollow fibers comprise micropores and a permeable material extending over the micropores.

9. The catheter system of claim 1 further comprising a trocar, the trocar comprising:
an elongated main body comprising a first end and a second end, the first end comprising a tip and the second end comprising a connector configured to connect to the proximal end of the catheter main body; and
a sheath surrounding the main body and slidable on the main body, the sheath comprising:
a neck portion surrounding a portion of the main body and having a circular cross-sectional shape,
a shoulder portion which flares outward in one plane to form an expanding oval cross sectional shape, and
a tubular sleeve portion extending around and spaced apart from the main body and having a uniform oval cross sectional shape,
wherein the shoulder portion is located between the neck portion and the tubular sleeve portion.

10. The catheter system of claim 9 wherein the shoulder portion flares outward at an angle of between about 30 degrees and about 60 degrees.

11. The catheter system of claim 9 wherein the connector is configured to fit snugly within the central lumen of the catheter main body.

12. The catheter system of claim 11 wherein the connector comprises a series of tapered barbs.

13. The catheter system of claim 9 wherein the elongated main body of the trocar comprises two straight portions connected at an angle.

14. The catheter system of claim 13 wherein the sheath is located between the angle and the connector of the main body.

15. A method of treatment of cerebral edema comprising:
inserting a distal end of a catheter into a ventricle of a brain of a patient suffering from cerebral edema, the catheter comprising:
an elongated main body comprising a proximal end, a distal end, a central lumen and a plurality of outer lumens, the central lumen and the outer lumens extending therethrough and the outer lumens positioned around the central lumen;
a hollow fiber portion distal to the main body, the hollow fiber portion comprising
a plurality of hollow fibers comprising elongated hollow tubes with semipermeable walls, the semipermeable walls being gas permeable and liquid impermeable, the hollow fibers each comprising a proximal end connected to one of the plurality of outer lumens, and
a drainage catheter, and the drainage catheter comprising a central lumen, a plurality of pores, the drainage catheter having a proximal end connected to the central lumen of the elongated main body and a distal sealed end, the plurality of hollow fibers are positioned around the drainage catheter;
a cap distal to and connected to the hollow fibers, the cap comprising an outer wall that forms a central space into which the hollow fibers extend and terminate at open distal ends, the central space forming a plenum for gas sweeping through the outer lumens and the hollow fibers; and
a manifold connected to the proximal end of the main body, wherein the outer lumens split into a first group and a second group lateral to the central lumen;
connecting the catheter to a source of dry gas and a vacuum, the dry gas supplied to the catheter through the first group of outer lumens and the vacuum applied to the catheter through the second group of outer lumens;
supplying the dry gas and vacuum to the catheter.

16. The method of claim 13 wherein the dry gas and the vacuum are supplied continuously.

17. The method of claim 13 wherein the dry gas and the vacuum are supplied intermittently.

18. The method of claim 13 further comprising draining cerebrospinal fluid from the ventricle through the central lumen of the main body.

19. The method of inserting a treatment catheter into a treatment location in a patient, the method comprising:
forming a craniotomy hole at a location in the skull of the patient;
inserting a first end of a treatment catheter through the craniotomy hole and to a position for treatment, the treatment catheter further comprising a second end which remains external to the patient after this step of inserting the first end;
forming a tunnel beneath the scalp of a patient, the tunnel extending from the location of a craniotomy hole to an incision in the scalp at a distance of 2 centimeters or more from the craniotomy hole location;
after inserting the first end of the treatment catheter through the craniotomy hole into the position for treatment, attaching the second end of a treatment catheter to a connector end of a trocar;
passing the trocar with the attached treatment catheter through the tunnel such that the trocar pulls the second end of the treatment catheter through the tunnel and the second end of the treatment catheter exists the tunnel through the incision; and then
detaching the trocar from the treatment catheter.

20. The method of claim 19, wherein the second end of the catheter further comprises an external aperture connected to a catheter lumen, the external aperture sealed with a removable plug, the method further comprising:
after the second end of the treatment catheter exists the tunnel through the incision, removing the plug from the aperture.
